(19) United States
(12) Patent Application Publication
(10) Pub. No.: US 2010/0311103 A1
(43) Pub. Date: Dec. 9, 2010

(54) SOLID SUPPORT COATED WITH AT LEAST ONE METAL FILM AND WITH AT LEAST ONE TRANSPARENT CONDUCTIVE OXIDE LAYER FOR DETECTION BY SPR AND/OR BY AN ELECTROCHEMICAL METHOD

(75) Inventors: Rabah Boukherroub, Villeneuve D'Ascq (FR); Xavier Castel, Lehon (FR); Sabine Szunerits, Grenoble (FR)

Correspondence Address: FOLEY AND LARDNER LLP;SUITE 500, 3000 K STREET NW, WASHINGTON, DC, 20007, US

(73) Assignees: CENTRE NATIOAL DE LA RECHERCHE SCIENTIFIQUE (CNRS); UNIVERSITE DES SCIENCES ET TECHNOLOGIES DE LILLE; INSTITUT POLYTECHNIQUE GRENOBLE; UNIVERSITE DE RENNES 1

(21) Appl. No.: 12/735,041

(22) PCT Filed: Dec. 11, 2008
(86) PCT No.: PCT/EP2008/067356

§ 371 Date: Jun. 11, 2010

(30) Foreign Application Priority Data
Dec. 11, 2007 (FR) 0759748

(51) Int. Cl. G01N 27/26; B32B 9/00; B32B 15/04; C23C 14/34; G01N 33/44; G01N 33/00; G01N 33/68; G01N 33/92; G01N 33/84; G01N 33/48; C12Q 1/02; C12M 1/34; G01N 21/55

(52) U.S. Cl. 435/29; 428/702; 428/697; 428/336; 428/469; 204/192.15; 436/85; 436/94; 436/86; 436/145; 436/71; 436/63; 205/787; 205/780.5; 205/777.5; 204/403.01; 422/98; 435/287.1; 356/445

(57) ABSTRACT

One subject of the present invention is a transparent solid support coated with at least one layer of metal and with at least one layer of transparent conductive oxide (TCO), especially tin-doped indium oxide (ITO) in order to form a solid support that can be used at the same time or independently for detection by SPR and by an electrochemical method. The invention comprises a process for producing such supports, especially by cathode sputtering using a device comprising a radiofrequency (RF) generator, this device also being included in the invention. Another subject of the invention is a kit and a method for detection or identification of an organic or mineral compound by surface plasmon resonance (SPR) and/or electrochemical plasmon resonance comprising or using such supports.

The present invention concerns a transparent solid support coated with at least one layer of metal and with at least one layer of transparent conductive oxide (TCO), in particular tin-doped indium oxide (ITO), to form a solid support that can be used at the same time or independently for detection by SPR and by an electrochemical method. The invention comprises a process for producing such supports, especially by depositing thin films by cathode sputtering using a device comprising a radiofrequency (RF) generator, this device also being included in the invention. The invention also concerns a kit and a method for detection or identification of organic or mineral compounds by surface plasmon resonance (SPR) and/or electrochemical plasmon resonance comprising or using such supports.

Surface plasmon resonance (SPR) is a sensitive spectroscopic technique that has now proven its usefulness as an analytical tool for monitoring interfacial processes or for characterizing thin films (1-3). The SPR technique allows detection without marking. The selectivity of the method comes from the stimulation of the electromagnetic fields at the metal-dielectric interface owing to the surface plasmons created. These surface plasmons are excited on metal surfaces (gold, for example) when a polarized light p illuminates the metal/dielectric interface via a prism whereof the total reflection is adjusted, coupling, from certain angles, the incident light with the surface plasmon modes. The formation of the plasmon is shown by the marked attenuation of the intensity of the reflected light (measured by a photodiode) at a certain incidence angle value Θ, noted resonance angle Θ_{SPR}. The position of this peak Θ_{SPR}, its minimum reflection factor Rₘᵢₙ as well as its width at mid-height (noted FWHM for “Full Width at Half Maximum”) are extremely sensitive to any variations in the refraction index (n) of the adjacent medium and its optical depth.

The choice of metal film in contact with the dielectric medium is a critical parameter. As the metal film is an absorbent medium at the working wavelength, the refraction index of the metal layer considerably influences the characteristics of the absorption peak of the plasmon curve (4-7). Appropriate metals include in particular silver, gold, copper and aluminum (4, 8, 9). Silver provides a much narrower or pointed SPR signal than that of gold (the full width at half maximum is 10.67° for gold and 0.71° for silver in the air (10)). The origin of this result is the low value of the real part n′ and the high value of the imaginary part n″ of the refraction index n (n=n′+in″) of the metal. Silver is also described as having increased sensitivity to the variations of the refraction index and optical depth relative to gold (7, 9-11). Moreover, while the penetration length of the evanescent wave produced by a gold film 50 nm thick is 164 nm with a light source adjusted to λ=630 nm, that of a silver film with the same depth is 219 nm (12). Although the silver appears to be the ideal candidate as metal film in a SPR chip, gold is more used because it has stable chemical and optical properties compared to those of silver. Indeed, the surface of silver is chemically unstable in reactive mediums. Exposed to air, silver oxidizes quickly, this oxidation being accelerated in aqueous solution. This is why the use of the silver interface/adjacent medium cannot be considered for optical measurements, also long-lasting, essential during monitoring of reactions and biological interactions (13). The use of a silver film therefore implies that its surface is covered with a uniform protective layer in order to preserve its advantageous optical properties. Different approaches have been considered for this protection; for example: the additional deposition of organic monofilm making it possible to obtain both an interface sensitive to the ion-H⁺ interaction and allowing near field coupling, as with polyionene (14) or aluminum tris-(8-hydroxyquinoline) (15).

Today, the SPR measurement technique is widely used for real-time detection of molecular and biomolecular events such as the study of protein-DNA interaction, DNA-DNA interaction, cell adhesion, DNA hybridization reactions.

The chemistry used for fixing biological components to the surface of the gold film of the chip (or SPR support) is primarily based on the use of thiolated compounds (16-21), conductive polymers (22-24) or a monolayer of functionalized dextran (Biacore system) (25). One can also cite the coupling chemistry of silane used with oxides allowing the fixing of the biomolecules. The process then consists of covering the noble metal, such as gold, with a fine layer of silicon oxide SiOx. One can cite in particular the processes for manufacturing a solid support coated with a layer of gold on which a layer of SiOx of uniform and stable depth has been deposited using the plasma-enhanced chemical vapor deposition (PECVD) technique (26-27). Although these films of SiOx are interesting for electrochemical measurements, nevertheless remain very resistive.

Thus, in light of the preceding, it would be desirable to be able to have a process making it possible to modify a solid support adapted to SPR measurement, with the aim of obtaining a solid support that can also be used for electrochemical measurements while broadening the electroactive window usually used. Preferably, it would be advantageous for this modification made to the SPR support also to be able to significantly improve the stability of its interface if this support was, for example, provided with a silver or copper metal film, while also keeping its advantageous optical properties compared to those of gold (for example: analysis depth doubled in the reactive medium; measurement window Δn of the refractive index of the adjacent medium increased). Also preferably, it would be advantageous for this modification made to the SPR support to also be able to facilitate covalent coupling of organic compound(s) at its surface.

This is the object of the present invention.

The inventors have shown that depositing a thin film of a transparent conductive oxide (TCO), such as tin-doped indium oxide (ITO), on a support adapted to SPR detection having a metal layer of gold, silver or copper on its surface, made it possible to obtain such a support that could be used at the same time or independently for detection by SPR and by an electrochemical method.

The ITO deposited in a thin layer is known to be widely used as electrode both conductive for the electrical current and transparent in the visible and the near infrared regions. These thin layers are used in devices such as liquid crystal displays (LCD), organic light-emitting devices (OLED), solar cells, detectors, window, mirror or lens heating devices, heat absorption reflective layers, electrochrome devices, etc.

The ITO is an n-type degenerate semiconductor. Its broad forbidden band (>3.5 eV) explains its transparency in the visible region. The electrical conductivity of the ITO results from the contribution of load carriers (electrons) on levels close to the bottom of the conduction band, on one hand by creating oxygen holes, and on the other hand by substitution, in the crystalline structure, of indium ions by tin ions.

Thus, under a first aspect, the present invention concerns a solid support characterized in that it comprises a transparent solid support coated in part or in whole with:

at least one layer of at least one metal to form a solid support that can be used for detection by SPR;

at least one layer of transparent conductive oxide (TCO);

and, if applicable, an attachment layer.

Preferably for the solid support according to the invention, said TCO layer makes it possible to form a solid support that can be used for detection by SPR and/or for electrochemical detection, also preferably to form a solid support that can be used for detection by SPR and for electrochemical detection.

Thus, for said TCO layer to have this conductive nature, it will preferably be chosen among the group of compounds made up of In₂O₃₋ₓ with 0<x<3; ZnO₁₋ₓ with 0<x<1; SnO₂₋ₓ with 0<x<2; CdO₁₋ₓ with 0<x<1; Ga₂O₃₋ₓ with 0<x<3; Tl₂O₃₋ₓ with 0<x<3; PbO₂₋ₓ with 0<x<2; Sb₂O₅₋ₓ with 0<x<5; MgO₁₋ₓ with 0<x<1 and TiO₂₋ₓ with 0<x<2, the values of x of the intervals being those making it possible to obtain a good, or even better conductance, in addition to the transparent nature of this oxide layer.

A transparent material is a material whereof the ratio between the light intensity passing through the material and the incident light intensity on the material is not zero in at least one range of wavelengths.

In one preferred embodiment, the solid support according to the invention is characterized in that said transparent conductive oxide (TCO) layer has a defined and stable depth.

In one preferred embodiment, the solid support according to the invention is characterized in that said TCO layer comprises at least one transparent conductive oxide preferably chosen from the group of In₂O₃; ZnO, SnO₂; CdO; Ga₂O₃; Tl₂O₃; PbO₂; Sb₂O₅; MgO; TiO₂.

In one preferred embodiment, the solid support according to the invention is characterized in that said TCO layer comprises at least one transparent conductive oxide made up of a combination of at least two binary oxides.

In one preferred embodiment, the solid support according to the invention is characterized in that said TCO layer also comprises a component capable of doping the TCO.

In one preferred embodiment, the solid support according to the invention is characterized in that said TCO layer is a layer comprising indium oxide In₂O₃.

In one preferred embodiment, the solid support according to the invention is characterized in that said TCO layer is a layer comprising tin-doped indium oxide (ITO), preferably synthesized from a target material made up of a mixture 90% In₂O₃ and 10% SnO₂ by mass.

In one preferred embodiment, the solid support according to the invention is characterized in that said TCO layer is a layer comprising tin-doped indium oxide (ITO) deposited at ambient temperature and with a mainly amorphous structure.

In one preferred embodiment, the solid support according to the invention is characterized in that said TCO layer has a depth between 3 nm and 200 nm, preferably the depth is between 2 nm and 20 nm, between 4 nm and 10 nm being the most preferred values. For example: 4 nm is the most preferred value for SPR supports made up of at least one gold film and used for detection by SPR and/or by an electrochemical method; 4 nm is the most preferred value for SPR supports made up of at least one silver film and used for detection by SPR; 10 nm is the most preferred value for SPR supports made up of at least one silver film and used for detection by SPR and/or by an electrochemical method.

In one preferred embodiment, the solid support according to the invention is characterized in that said layer made up of at least one metal is a layer whereof the metal is chosen in the group made up of gold, silver, copper and aluminum or by any combination of these metals or of their respective alloys.

In one particular embodiment, said layer of at least one metal is made up of or comprises metal nanoparticles, preferably whereof the diameter is between 2.5 nm and 100 nm.

In one preferred embodiment, the solid support according to the invention is characterized in that said layer made up of at least one metal has a depth between 10 nm and 200 nm, preferably between 30 and 50 nm.

In one preferred embodiment, the solid support according to the invention is characterized in that said solid support is coated with an attachment layer before said layer made up of at least one metal, preferably with a depth between 1 nm to 10 nm, 5 nm±1 nm being the preferred depth.

In one preferred embodiment, the solid support according to the invention is characterized in that said attachment layer is a metal layer whereof the metal is chosen from the group made up of titanium, chrome, nickel, tantalum, molybdenum, thorium, copper, aluminum or tin or by any combination of these metals or of their respective alloys, oxides and/or hydroxides.

In one preferred embodiment, the solid support according to the invention is characterized in that said attachment layer is a metal oxide MOx layer, with oxygen gradient, with M designating at least one metal chosen from the group of gold, silver, copper and aluminum, or by any combination of these metals or of their respective alloys.

In one particularly preferred embodiment, the solid support according to the invention is characterized in that said attachment layer is preferably a layer of titanium.

In one preferred embodiment, the solid support according to the invention is characterized in that said solid support is made up of at least one organic or inorganic transparent material or a combination of transparent materials such as glass or transparent solid polymers such as polymethylpentene (TPX), polyethylene, polyethylene terephthalate (PET), polycarbonate.

Said solid support is preferably chosen in glass.

In one particular embodiment, the solid support according to the invention is characterized in that said layer of at least one metal to form a solid support that can be used for detection by SPR is a layer made up of metal nanoparticles, preferably chosen among the supports shown in FIGS. 20 and 21.

In one particular embodiment, the metal film comprising said metal nanoparticles, preferably gold or silver, is obtained by evaporation.

In another preferred embodiment, the solid support according to the invention is characterized in that said transparent conductive oxide (TCO) layer is coated with a layer made up of metal nanoparticles, preferably as shown in FIG. 17, preferably the metal particles are gold or silver. Also preferably, this metal film comprising these metal nanoparticles is obtained by evaporation of a second metal film to form metal nanoparticles on the TCO layer, preferably said second metal film has a depth less than 10 nm, preferably less than 5 nm (see also PCT patent application Boukherroub et al. published under number WO 2007/036544 for the realization of a metal film of metal nanoparticles).

In still another particular embodiment, the solid support according to the invention is characterized in that said transparent conductive oxide (TCO) layer is coated with a layer made up of metal nanoparticles, the latter layer of metal nanoparticles itself being coated with a TCO layer, preferably as shown in FIG. 18 or 19 (multilayers in FIG. 19 n preferably being between 2 and 10 (inclusive), more preferably equal to 2, 3, 4 or 5).

Obtaining, on a solid support, a metal film made up of or comprising metal nanoparticles, preferably gold or silver, is well known by those skilled in the art and will not be developed here. Non-limitingly, one can cite, for example, the direct deposition of commercially available metal nanoparticles, with a diameter preferably between 2.5 nm and 100 nm, on the glass or metal oxide surface (the glass surface being able to be silanized beforehand to ultimately present groups of positive charges for better fixing of those particles (in general presented in monodispersed form and with negative charges). One can also cite the obtaining of such a layer thermally (evaporation/curing of a metal film with small depth), or by electrochemical deposition on an ITO-type conductive film.

In one preferred embodiment, the solid support according to the invention is characterized in that at least one TCO layer has hydroxyl groups.

Preferably, the hydroxyl groups are chemically activated so as to be able to bind through covalent bonding to reactive groups such as silanes, if applicable, these silane groups themselves being functionalized by groups chosen from the thiol, amine, acid, cyanide, aldehyde groups, electrochemically active, photoactivable, etc. groups, but also with other molecules carrying active functionalities for the hydroxyl groups (acid, amine, etc.).

The TCO surfaces, in particular ITO, having activated hydroxyl groups can be used for the anchoring, by covalent coupling, of silane compounds functionalized with thiol groups. These thiol groups can then easily form a disulfide bridge with a bifunctional reagent having a thiol function and a terminal amine function (for example by reaction with the 2-(2-pyridinyldithio)ethanamine hydrochloride compound). This amine group is then used to fix a curing agent (crosslinker) there, the latter being chosen to be capable of fixing the chosen probe, for example DNA or a protein, for a given detection method. The advantage of the presence of a disulfide bridge is to be able to reuse or recycle the support after eliminating the probe through a reduction reaction (28).

For the surface chemistry making it possible to graft biomolecules such as proteins or nucleic acids, on the ITO layer having activated hydroxyl groups, one can also cite the method by which one performs the deposition of the (N-(2-aminoethyl)-3-aminopropyl-trimethoxysilane (AEAPTS) compound on a glass support coated with at least one ITO layer using the chemistry of the N-ethyl-N′-(3-dimethylaminopropyl) carbodiimide and the N-hydroxysuccinimide (EDC/NSH)) (29). For grafting of nucleic acids (RNA or DNA), one can graft amino groups beforehand to one of the ends of these nucleic acids and come back to use this same type of reagent (EDC/NHS).

In one preferred embodiment, the solid support according to the invention is characterized in that said hydroxyl groups and/or the functional groups having reacted with said hydroxyl groups can be desorbed from said TCO layer by exposure to ultraviolet radiation or by chemical reduction.

Under a second aspect, the present invention concerns a method for manufacturing a solid support for detection by surface plasmon resonance (SPR) and by electrochemical methods, characterized in that it comprises the following steps:

the deposition on at least a same surface of the solid support and superimposed,

of at least one layer made up of at least one metal to form a solid support that can be used for detection by SPR; and

of at least one transparent conductive oxide (TCO) layer, preferably said TCO layer is made up of at least one transparent conductive oxide (TCO) layer to form a solid support that can be used for detection by SPR and/or for electrochemical detection, preferably able to be used for detection by SPR and for electrochemical detection.

The transparent conductive oxide (TCO) layer is deposited here to (i) facilitate the covalent grafting of organic compound(s); (ii) form a solid support that can also be used for electrochemical measurements; (iii) protect, if necessary, the metal layer from any outside attack(s).

In one preferred embodiment, the process according to the invention is characterized in that the deposition of the TCO layer is done in a vacuum chamber, preferably provided with a residual pressure between 10⁻⁵ and 10⁻⁷ mbar or less.

In one preferred embodiment, the process according to the invention is characterized in that the deposition of the TCO layer is done in partial vacuum in the presence of at least one rare gas, preferably argon, or in the presence of a mixture of a rare gas (preferably argon) and a gas containing the oxygen element (preferably dioxygen), preferably at a pressure of about 0.009 torr of rare gas/dioxygen mixture, preferably with a p_{O2}/p_{Ar} ratio equal to about 5.1.10⁻⁴.

In one preferred embodiment, the process according to the invention is characterized in that the TCO layer is deposited on the solid support by cathode sputtering in that the cathode sputtering housing comprises at least one generator, preferably a radiofrequency (RF) generator, and in that the radiofrequency power used for the deposition is calculated as a function of the area of the target surface (source material of the TCO layer), the distance separating the target from the solid support (substrate), preferably between 0.1 W/cm² and 4 W/cm² for a distance between 10 mm and 150 mm, preferably 0.86 W/cm² for a target-substrate distance of 78 mm.

In one preferred embodiment, the process according to the invention is characterized in that the depth of said TCO layer is controlled by the duration of the deposition, preferably at a speed of 0.6 nm/min at 0.86 W/cm² for a target/substrate distance of 78 mm.

It has been shown that the deposition by cathode sputtering (FIG. 1) was a method of choice for the synthesis of such layers. Their synthesis is, in general, associated with a high-temperature treatment (≈400° C.) that produces polycrystalline layers, either by heating the substrate during the deposition, or by performing curing in a controlled atmosphere after the deposition. This is not the object of the embodiment of the present invention.

On the contrary, we have recently shown the possibility of depositing, at ambient temperature, such transparent conductive layers on temperature-fragile substrates, such as plastic films having small radiofrequency or hyperfrequency losses, for example on transparent plastic polymers such as Zeonex® (copolyolefin manufactured by NIPPON ZEON), polymethylpentene (TPX), Transphan® (cyclic olefin polymer having a high vitreous transition temperature available from Lofo High Tech Film, GMBH, Germany) or Arton G® or Artong® (manufactured by the company Japan Synthetic Rubber Co., Tokyo, Japan).

In one preferred embodiment, the process according to the invention is characterized in that said TCO layer comprises at least one transparent conductive oxide chosen from the group made up of In₂O₃₋ₓ with 0<x<3; ZnO₁₋ₓ with 0<x<1; SnO₂₋ₓ with 0<x<2; CdO₁₋ₓ with 0<x<1; Ga₂O₃₋ₓ with 0<x<3; Tl₂O₃₋ₓ with 0<x<3; PbO₂₋ₓ with 0<x<2; Sb₂O₅₋ₓ with 0<x<5; MgO₁₋ₓ with 0<x<1 and TiO₂₋ₓ with 0<x<2, preferably chosen among In₂O₃; ZnO, SnO₂; CdO; Ga₂O₃; Tl₂O₃; PbO₂; Sb₂O₅; MgO; TiO₂.

In one preferred embodiment, the process according to the invention is characterized in that said TCO layer comprises at least one oxide made up of a combination of at least two binary oxides.

In one preferred embodiment, the process according to the invention is characterized in that said TCO layer comprises any combination of the TCOs with a component capable of doping those TCO such as Sn for In₂O₃.

In one particularly preferred embodiment, the process according to the invention is characterized in that said TCO layer is a layer comprising indium oxide In₂O₃.

In one even more particularly preferred embodiment, the process according to the invention is characterized in that said TCO layer is a layer comprising tin-doped indium oxide (ITO), preferably synthesized from a target material made up of a mixture of 90% In₂O₃ and 10% SnO₂ by mass.

In still another preferred embodiment, the process according to the invention is characterized in that said TCO layer is a layer comprising tin-doped indium oxide (ITO) deposited at ambient temperature and with a mainly amorphous structure.

In one preferred embodiment, the process according to the invention is characterized in that said TCO layer has a depth between 3 nm and 200 nm, preferably the depth is between 3 nm and 20 nm, between 4 nm and 10 nm being the most preferred values. For example: 4 nm is the most preferred value for SPR supports made up of at least one gold film and used for detection by SPR and/or by an electrochemical method; 4 nm is the most preferred value for SPR supports made up of at least one silver film and used for detection by SPR; 10 nm is the most preferred value for SPR supports made up of at least one silver film and used for detection by SPR and/or by an electrochemical method.

By cathode sputtering and without heating the substrate, we synthesized mainly amorphous ITO layers, but having electrical conduction and transparency characteristics comparable to those of polycrystalline ITO layers (30, 31). The choice of a set of sputtering conditions comprising a high pressure, a continuous current power (DC) or modest radiofrequency (RF), and a great target-substrate distance, favors the thermalization of the atoms arriving on the substrate, and therefore favors the obtaining of an amorphous layer (31-33).

Moreover, it has been shown that tin does not contribute to the creation of carriers in amorphous ITO, such that what is said about amorphous ITO can also be applied to pure and amorphous In₂O₃ (34-36).

To obtain optimal properties of the In₂O₃ or the amorphous ITO, the partial oxygen pressure in the working atmosphere is critical (FIG. 2). It depends on other sputtering parameters (nature of the target, use of a magnetron, DC or RF power, target/substrate distance, etc.). It has been determined empirically (30). Under these conditions and for small depths d the layers have an amorphous or very slightly polycrystalline structure (FIG. 3), a smooth surface (FIG. 4) and their resistivity ρ is constant as a function of the depth d (FIG. 5).

The layers obtained do not require any treatment after coming out of the enclosure, and they are stable at ambient up to a temperature of about 125-180° C., after which temperature they begin to crystallize with notable speed, in our experience and according to various authors (35, 37, 38). This threshold temperature is compatible with many applications, in particular when the substrate is organic, and therefore itself temperature-fragile. Moreover, it is probable that the absence of curing limits surface reconstruction phenomena of the oxide (39) and that it preserves better reactivity, useful, in particular, for applications of the molecular grafting type. Moreover, ITO being a sub-stoichiometric oxide, the molecular grafting is all the more simple relative to (i) a surface made up of precious metals (Au) or semi-precious metals (Ag, . . . ) and (ii) opens a new path for chemisorption (chemical surface structuring using electroactive organic layers, for example).

The good conductance of the deposited oxide is also crucial for the electrical contacts on the multilayer when an electrochemical measurement or detection is contemplated (in parallel to the surface plasmon detection).

In one preferred embodiment, the process according to the invention is characterized in that said layer made up of at least one metal is a layer whereof the metal is chosen in the group made up of gold, silver, copper and aluminum or by any combination of these metals or of their respective alloys, gold, silver and copper being the most preferred.

In one preferred embodiment, the process according to the invention is characterized in that said layer made up of at least one metal has a depth between 10 nm and 200 nm, preferably between 30 and 50 nm.

In one preferred embodiment, the process according to the invention is characterized in that said solid support is first coated with an attachment layer.

In one preferred embodiment, the process according to the invention is characterized in that said attachment layer has a depth between 1 and 10 nm, before the deposition of said layer made up of at least one metal, preferably with a depth of 5 nm±1 nm.

In one preferred embodiment, the process according to the invention is characterized in that said attachment layer is a metal layer whereof the metal is chosen from the group made up of titanium, chrome, nickel, tantalum, molybdenum, thorium, copper, aluminum, tin, or by any combination of these metals or of their respective alloys, oxides and/or hydroxides.

In one preferred embodiment, the process according to the invention is characterized in that said attachment layer is a layer of metal oxide MOx, with oxygen gradient, with M designating at least one metal chosen in the group made up of gold, silver, copper and aluminum, or by any combination of these metals or of their respective alloys.

In one particularly preferred embodiment, the process according to the invention is characterized in that said attachment layer is a layer of titanium.

In one preferred embodiment, the process according to the invention is characterized in that said solid support is first coated with an attachment layer and said layer made up of at least one metal, before the deposition of said TCO layer.

In one preferred embodiment, the process according to the invention is characterized in that if necessary, said attachment layer and said layer made up of at least one metal is (are) also deposited by cathode sputtering.

The interest of the invention also lies in the absence of any temperature transition of the ITO/metal/ . . . layer, the problems of diffusion and/or delamination between layers are avoided whereas they are encountered by others with multilayers based on polycrystalline ITO (40-42).

In one preferred embodiment, the process according to the invention is characterized in that, if necessary, said attachment layer, said layer made up of at least one metal and said TCO layer are successively deposited on said solid support by cathode sputtering within a same device comprising an enclosure provided with a system of at least two targets, one of which is made up of the metal or alloy used to develop said layer made up of at least one metal, and the other of which is made up of the material used to develop said TCO layer, and, if applicable, of a target made of metal or alloy used to develop said attachment layer, and, if necessary, any useful target, said device being provided with at least one vacuum pump to create a partial vacuum in the enclosure and at least one controlled inlet for rare gas, preferably argon, and, if necessary, additional controlled inlets for reactive gas(es), preferably a carrier gas of the oxygen element, preferably dioxygen, and/or of at least one controlled inlet for a pre-established mixture of rare gas and reactive gas(es), preferably a carrier gas of the oxygen element, preferably dioxygen.

In one preferred embodiment, the process according to the invention is characterized in that said solid support is made up of at least one organic or inorganic transparent material or a combination of transparent materials.

Preferably, said transparent material is chosen among glass or transparent solid polymers such as polymethylpentene (TPX), polyethylene, polyethylene terephthalate (PET), polycarbonate, in particular when the manufacturing method uses the cathode sputtering technique at ambient temperature, glass being the most preferred solid support.

Under a third aspect, the present invention concerns a process for determining at least one organic or mineral compound in a sample or for monitoring at least one reaction in a complex mixture by SPR and/or electrochemical detection, characterized in that it uses a solid support according to the invention or capable of being obtained by a process according to the invention.

The invention also concerns the use of a support according to the invention or capable of being obtained by a process according to the invention for the detection in a sample of chemical or mineral compound(s), comprising in particular polymers or heavy metals, organic or biological compounds or structures comprising in particular nucleic acids, polypeptides or proteins, carbon hydrates, organic particles such as liposomes or vesicles, inorganic particles (such as micro- or nanospheres, cellular organelles or cells).

Under a fourth aspect, the present invention concerns a kit or supplies to determine the presence and/or quantity of at least one compound or to monitor at least one reaction in a sample by SPR and/or by electrochemistry, characterized in that it comprises a support according to the invention or capable of being obtained by a process according to the invention.

Under a last aspect, the invention concerns a diagnostic or analysis device comprising a support according to the invention or capable of being obtained using a process according to the invention.

Said device preferably comprises an enclosure provided with a system of at least two targets, one of which is made up of the metal or alloy used to develop said layer of at least one metal, and the other of which is made up of the material used to synthesize said TCO layer, and, if necessary, of a target made up of the metal or alloy used to develop said attachment layer, and if necessary, of any useful target as defined in one of the claims according to the invention, said device being provided with at least one vacuum pump to create a partial vacuum in the enclosure and at least one controlled intake for rare gas, preferably argon, and, if necessary, additional controlled intakes for reactive gas(es), preferably a carrier gas of the oxygen element, preferably dioxygen, and/or at least one controlled intake of a pre-established mixture of rare gas and reactive gas(es), preferably of a carrier gas of the oxygen element, preferably dioxygen.

Other features and advantages of the invention will appear in the continuation of the description with the examples and figures, the legends of which are shown hereinafter.

It will be understood by all practitioners experienced in the development of thin layers that the conditions cited in the examples below can vary (in particular depending on the equipment) without being detrimental to the interest of the invention.

### LEGEND FOR THE FIGURES

FIG. 1: RF cathode sputtering housing

FIG. 1 is a principle diagram of a cathode sputtering housing, used to deposit layers of oxide and/or metal layers. The supply of the target with radiofrequency voltage (as shown here, or DC high voltage) creates a discharge forming a plasma between the target (cathode) and the anode. The sputtering of the surface of the target by the Ar⁺ ions ejects the component atoms from the latter. The elements are thus ejected into the enclosure and a portion condenses on the substrate placed opposite.

Although we have only shown one ITO target, the housing advantageously has multiple targets to avoid breaking the vacuum between successive depositions of films of different natures.

FIG. 2: Evolution of the resistivity p as a function of the proportion of oxygen injected into the plasma during the ITO deposition.

FIG. 3: X-ray diffraction diagrams of an ITO layer deposited on a glass substrate, before (amorphous state) and after curing at 400° C. (polycrystalline state).

The bottom diagram, obtained before curing, shows at 2Θ≈25°, a diffusion peak due to the amorphous state of the glass substrate, on which at 2Θ≈31° another diffusion peak is superimposed that is characteristic of the indium oxide in amorphous state. One notes the presence of several peaks of very low intensity at 2Θ≈30.3°; 35.2°; 50.5° and 60.2° that are due to the diffraction of the cleavage planes of the indium oxide in the polycrystalline state (in extremely small quantity).

The upper diagram, obtained after curing of this same sample, was offset upwardly for better readability. The diffusion peak of the substrate still exists, the diffusion peak of the In₂O₃ around 31° has completely disappeared, and the diffraction peaks of the cleavage planes are exalted.

These diagrams demonstrate that the curing has completely converted the raw ITO layer of essentially amorphous deposition into a polycrystalline layer without preferential orientation.

FIG. 4: SEM examination of the surface state of an ITO layer with a depth of 200 nm.

Note the very smooth appearance of the surface morphology, also related to the amorphous state of the ITO.

FIG. 5: Conductance per square G□=d/ρ of ITO layers as a function of their depth d.

FIG. 5 shows several conductance values per square measured on different ITO layers, as a function of their depth. Up to a depth of about 200 nm, the conductance is proportional to the depth (square points).

FIG. 6: Load lock and RF cathode sputtering deposition enclosure (top view). The deposition enclosure is in multi-layer configuration: ITO-CU-Ti.

FIG. 7: Comparison of the transmittance of SPR chips covered or not covered with an ITO layer. Note the antireflection effect caused by the ITO layer.

FIG. 8: Inside of the deposition enclosure in multi-layer configuration: ITO-Ag—Ti.

FIG. 9: Reflectance as a function of the incidence angle Θ for different SPR supports immersed in water: (A) Ag (38 nm)/Ti (5 nm) (black), (B) ITO (4 nm)/Ag (38 nm)/Ti (5 nm) (gray) (C) Au (50 nm)/Ti (5 nm) (blue), (D) ITO (4 nm)/Au (40 nm)/Ti (5 nm) (green), experimental curves: dotted lines, theoretical SPR curves: solid lines; parameters used for the theoretical curves: see tables 3A and 3B.

FIG. 10: Variation of the resonance angle (ΔΘ_{SPR}) for 4 SPR supports immersed in water at ambient temperature for 2 hours: (A) Ag (38 nm)/Ti (5 nm), (B) ITO (4 nm)/Ag (38 nm)/Ti (5 nm), (C) Au (50 nm)/Ti (5 nm), (D) ITO (4 nm)/Au (40 nm)/Ti (5 nm).

FIGS. 11A-11D: Reflectance as a function of the incidence angle Θ for different SPR supports: (A) ITO (4 nm)/Ag (38 nm)/Ti (5 nm), (B) Au (50 nm)/Ti (5 nm), (C) ITO (4 nm)/Au (40 nm)/Ti (5 nm) and (D) ITO (4 nm)/Cu (44 nm)/Ti (5 nm); experimental curves: dotted lines, theoretical SPR curves: solid lines; parameters used for the theoretical curves: see tables 3A and 3B: water (black), ethanol (blue), hexane (red), 1-butanol (green), 2-pentanol (gray), 1-hexanol (orange), 1,3-propanediol (purple).

FIG. 12: Evolution of the resonance angle Θ_{SPR} of 5 SPR supports as a function of the refraction index for: ITO (4 nm)/Ag (38 nm)/Ti (5 nm) (solid circles), Au (50 nm)/Ti (5 nm) (solid squares) and ITO (4 nm)/Au (40 nm)/Ti (5 nm) (open squares); the theoretical values, given by the “Windspall” software, of Ag (38 nm)/Ti (5 nm) (open circles) and ITO (4 nm)/Cu (44 nm)/Ti (5 nm) solid triangles) were added for comparison.

FIGS. 13A-13B: (A) Variation of the resonance angle (ΔΘ_{SPR}) of the ITO/Ag/Ti support in contact with aqueous solutions of NiSO₄ of different concentrations (0.4.10⁻³ mol. L⁻¹ at 50.10⁻³ mol·L⁻¹), (B) Variation of the resonance angle ΔΘ_{SPR} as a function of the concentration in NiSO₄ for different SPR supports: ITO/Ag/Ti (open gray squares), Au/Ti (open circles), ITO/Au/Ti (solid circles).

FIG. 14: Voltammograms recorded with the Autolab apparatus with a reference electrode Ag/AgCl at the scanning speed of 50 mV/s in a solution of KCl of concentration 0.1 mol·L⁻¹. Measurements done with the supports: Au (50 nm)/Ti (5 nm) (black); ITO (4 nm)/Au (40 nm)/Ti (5 nm) (blue). The grafting of thiol groups to the surface of the Au/Ti support will show a localized peak at the arrow (−0.5 V).

FIG. 15: Voltammograms recorded with the Autolab 30 apparatus with a reference electrode Ag/AgCl and at the scanning speed of 50 mV/s in an aqueous solution containing a mixture of [Fe(CN)₆]⁴⁻ of concentration 10⁻² mol·L⁻¹ and Kcl of concentration 0.1 mol·L⁻¹. Measurements done with the supports: Au (50 nm)/Ti (5 nm) (black); ITO (4 nm)/Au (38 nm)/Ti (5 nm) (gray); ITO (10 nm)/Ag (38 nm)/Ti (5 nm) (gray dotted line).

FIG. 16: Reflectance as a function of the incidence angle Θ for supports modified by the electrochemical deposition of a film of 5 nm of polypyrrole: ITO (4 nm)/Ag (38 nm)/Ti (5 nm) (black), Au (50 nm)/Ti (5 nm) (red) and (c) ITO (4 nm)/Au (40 nm)/Ti (5 nm) (blue). Measurements done in a solution of LiClO₄ of concentration 0.1 mol·L⁻¹. Experimental curves: dotted lines; theoretical SPR curves: solid lines; parameters used for the theoretical curves: see tables 3A and 3B.

FIG. 17: FIG. 17 is a diagram showing a support produced by a deposition of metal nanoparticles on the TCO layer. The deposition can be done by cathode sputtering (in the same housing); by thermal evaporation, chemically or electrochemically.

FIG. 18: FIG. 18 is a diagram showing a support realized by a deposition of a TCO layer on the metal nanoparticles. The deposition can be done by cathode sputtering, PECVD, thermal evaporation, chemically or electrochemically.

FIG. 19: FIG. 19 is a diagram showing a support realized by a multi-layer deposition, layer by layer of metal nanoparticles and TCO.

FIG. 20: FIG. 20 is a diagram showing a support realized by a deposition of metal nanoparticles on glass. The deposition can be done by cathode sputtering, thermal, chemical or electrochemical evaporation.

FIG. 21: FIG. 21 is a diagram showing a support realized by a multi-layer deposition: layer by layer of metal nanoparticles and TCO.

### EXAMPLES

### Example 1

### Materials

Potassium chloride (KCl), potassium hexacyanoferricyanide (Fe(CN)₆⁻⁴), pyrrole, methanol, ethanol, hexane, 1-butanol, 2-propanol, 1-hexanol, 1,3-propanediol are obtained from Aldrich and used without additional purification.

### Example 2

### ITO Deposition on Au (40 nm)/Ti (5 nm) Bilayer (Bilayer Synthesized by Evaporation at the IEMN)

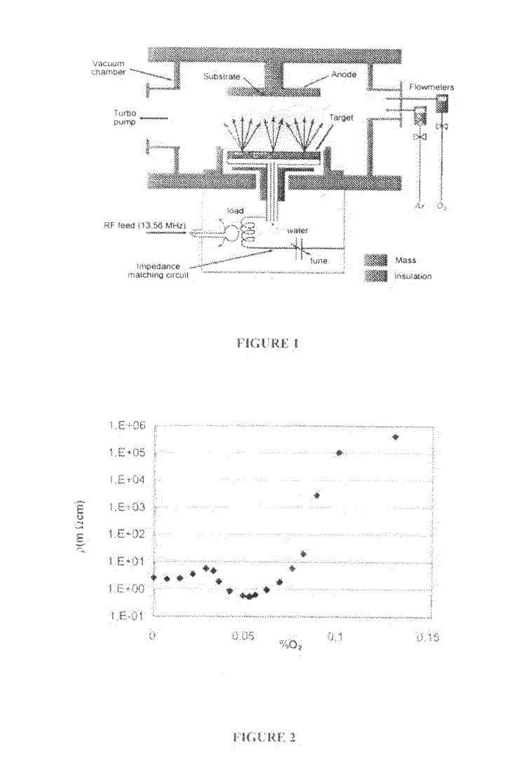

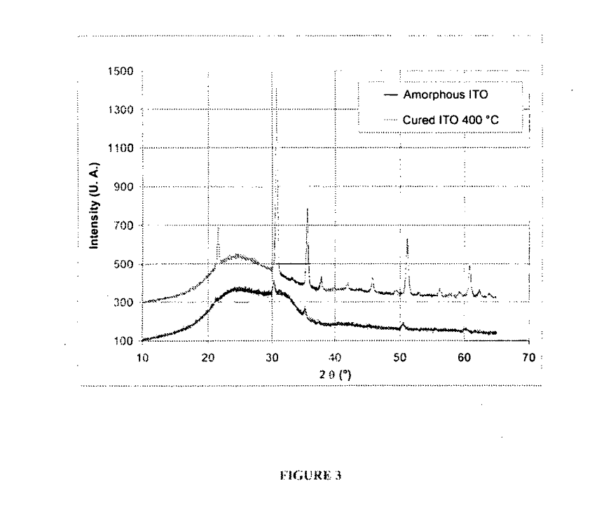

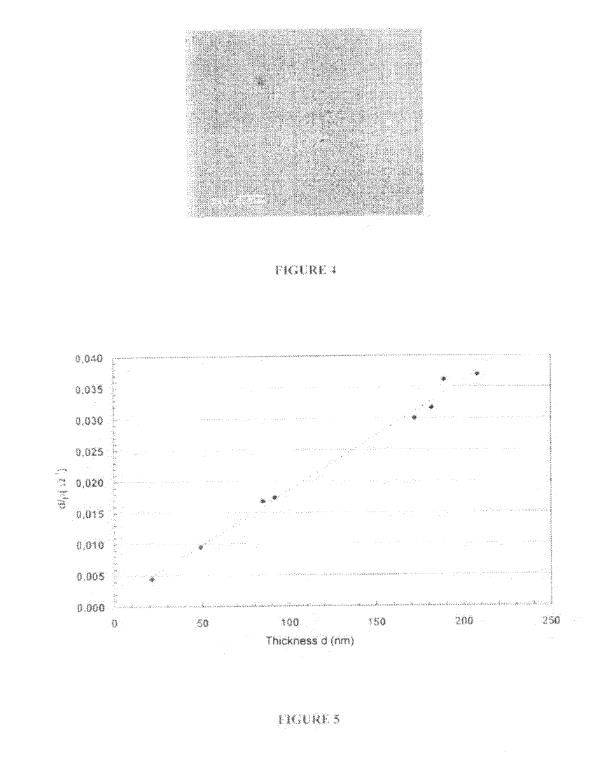

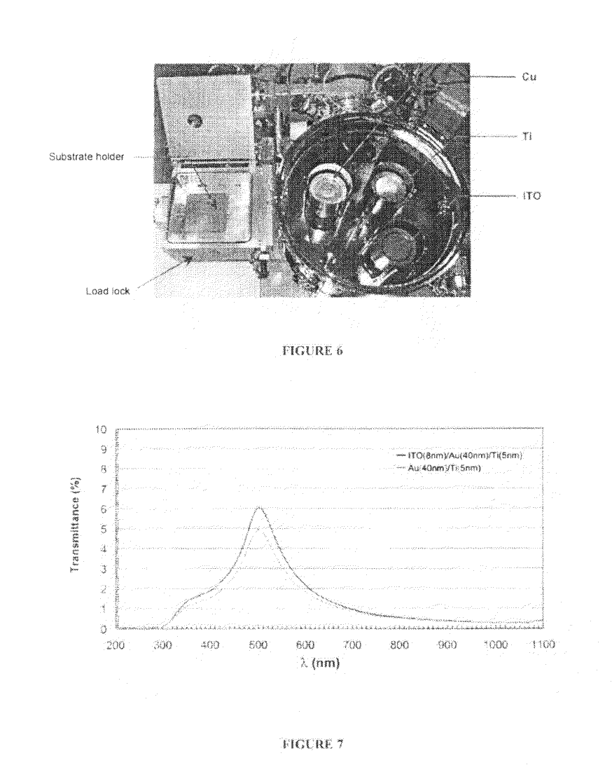

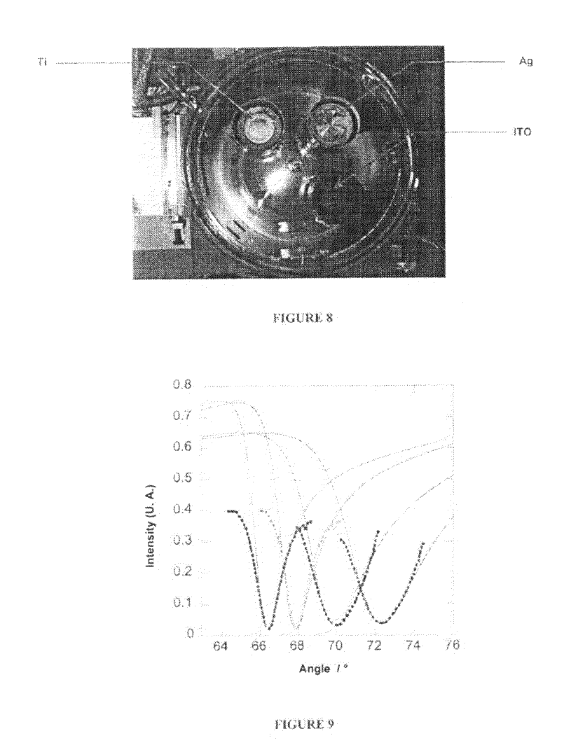

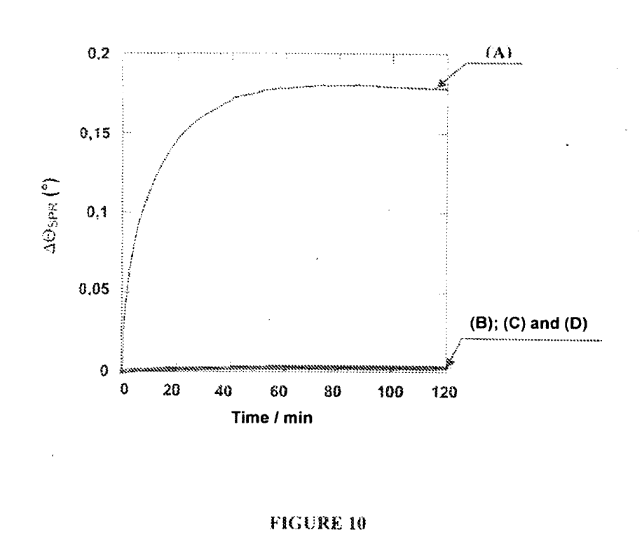

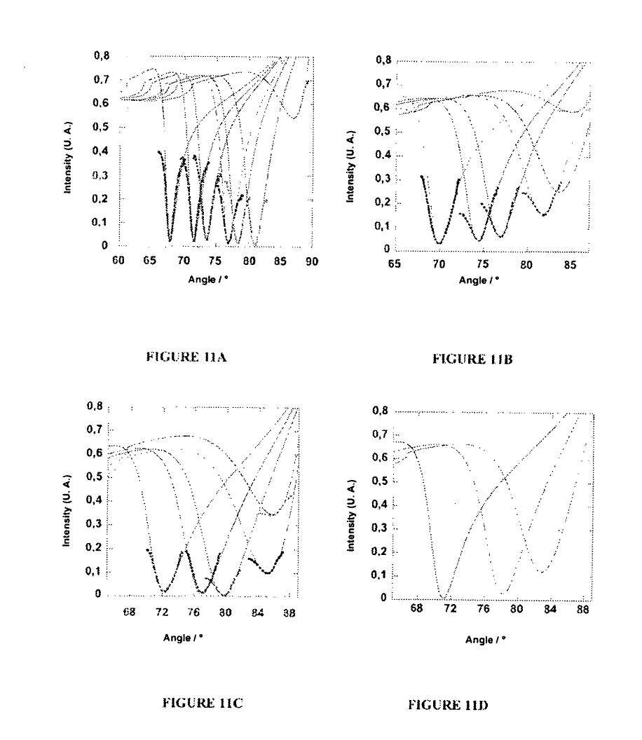

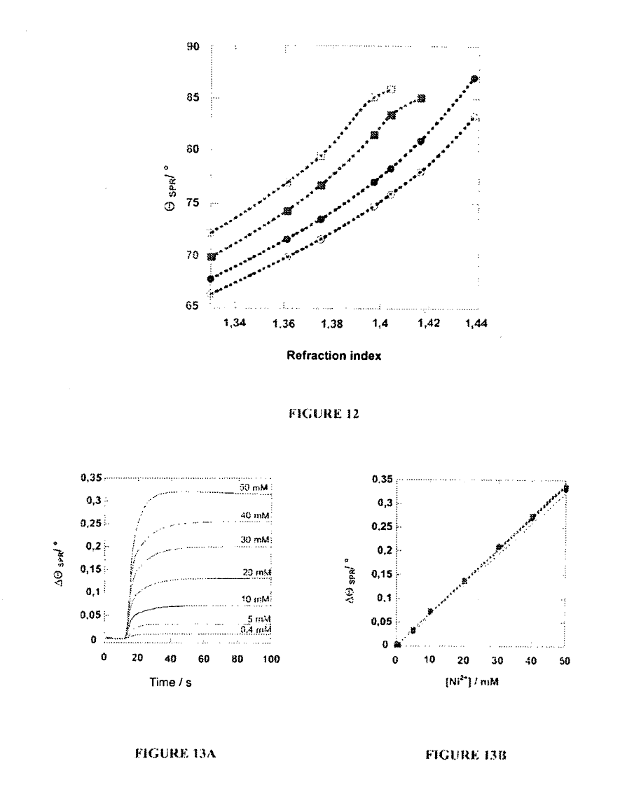

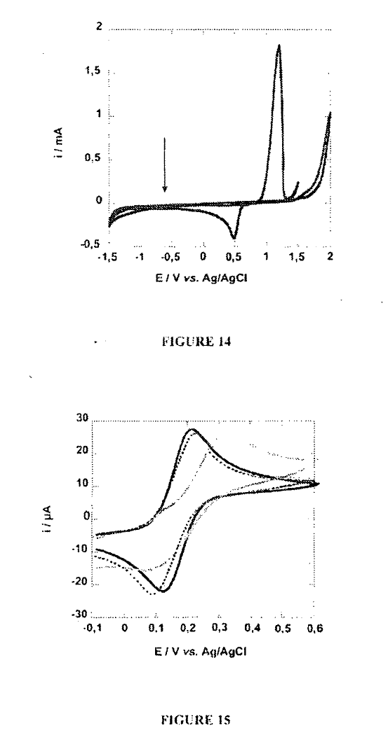

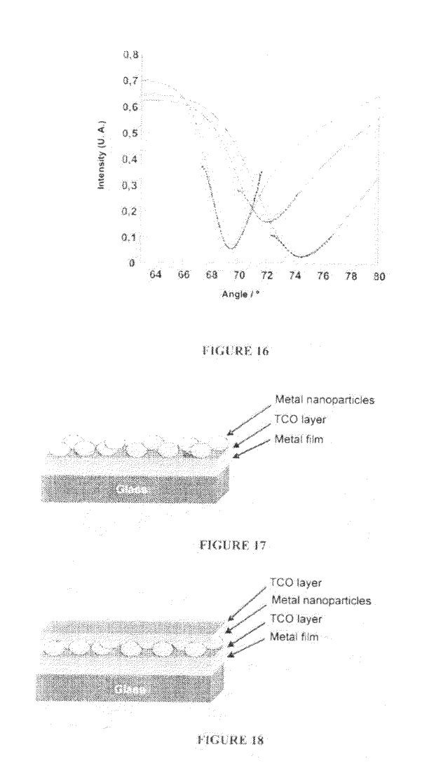

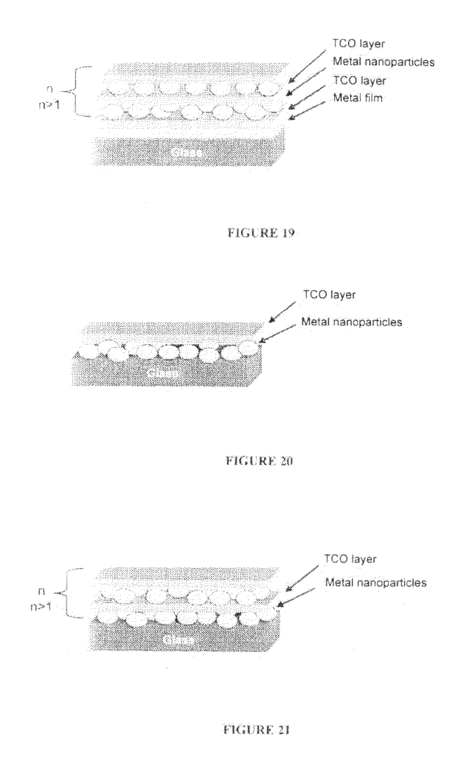

The thin film of ITO is deposited at ambient temperature by RF cathode sputtering from an ITO ceramic target (purity=99.999% and composition≈90% In₂O₃-10% SnO₂ by mass) of 75 mm in diameter, and in a newly cleaned and degassed enclosure, according to the rules of the art. The pumping is done by a turbomolecular pump, and the threshold pressure Pₒ in the enclosure is better than 5.10⁻⁷ mbar. The process gasses, Ar and O₂, are introduced during the deposition by two gas lines equipped with mass flowmeters, from pure gas bottles (FIG. 1).

The bilayer, after reception, is positioned directly on the substrate holder. It does not undergo any prior treatment. It is then introduced, via a load lock, into the deposition enclosure (FIG. 6).

After pumping to P≦10⁻⁶ mbar, the ITO target undergoes a pre-sputtering for 30 minutes (parameters grouped together in table 1) in order to free itself of any memory effect of the surface of the target related to the preceding deposition. The deposition of the ITO material on the bilayer is then done (parameters grouped together in table 2). The depth of the ITO film is controlled by the deposition time, after determining the deposition speed.

TABLE 1
pre-sputtering parameters of the ITO target

| | | | Ratio of | |
|---|---|---|---|---|
| | RF | Total | partial | |
| | Power | pressure | pressures | Target/substrate |
| | P_{RF} | Pₜₒₜ | P_{O2}/P_{Ar} | distance |
| Pre-sputtering | 65 W | 0.011 torr | 4.4.10⁻³ | 78 mm |
| ITO target | | | | |

TABLE 2
deposition parameters for the ITO material

| | | | Ratio | | |
|---|---|---|---|---|---|
| | | | of | | |
| | RF | Total | partial | Target/ | |
| | Power | pressure | pressures | substrate | Deposition |
| | P_{RF} | Pₜₒₜ | P_{O2}/P_{Ar} | distance | speed |
| ITO | 38 W | 0.009 torr | 5.1.10⁻⁴ | 78 mm | 0.6 nm/min |
| deposition | | | | | |

The technique for developing ITO films at ambient temperature makes it possible to perform the deposition on a presynthesized bilayer using another technique (evaporation in this case) without particular surface preparation. The mechanical resistance of the ITO on gold has been validated. No delaminating or interdiffusion effect between the 3 materials was revealed. The plasmon response of these samples was also validated.

In this example, the ITO layer is synthesized to:
facilitate covalent grafting of the organic compound(s);
carry out electrochemical measurements;
broaden the electroactive window for use of the gold for electrochemical measurements (see Example 6; C. Electrochemical measurements; point C.);
protect the fine layer of gold from mechanical attacks;
be used for the optical adaptation of the transmittance as a function of the wavelength (43), for example: the role of anti-reflection (FIG. 7) or interference filter. The optical properties of the stack may be optimized empirically, or calculated using the methods described—for example—in the class work by Macleod (44), or calculated by commercial software based on the same principles.

### Example 3

### ITO/Ag/Ti Trilayer Synthesis

Very narrow SPR peaks are obtained by using a metal provided with a low value n′ associated with a strong value n″ of its refraction index n (n=n′+in″). A “silver” SPR chip will therefore have a better theoretical sensitivity than a “gold” chip (table 3A), as previously manufactured. However, since silver oxidizes and/or sulfonates spontaneously whether in open air or by immersion in the solution to be analyzed, this type of chip (Ag/Ti) therefore cannot be used and consequently marketed, unless a protective layer is deposited on the surface of the layer of silver.

TABLES 3
(A) Complex refraction indexes for the
materials used to manufacture the different SPR supports
studied. (B) Refraction indexes for the different
dielectric mediums used. The refraction indexes are given
at λ = 670 nm.
Table 3A

| Material | n′ | n″ |
|---|---|---|
| Au | 0.197 | 3.67 |
| Ag | 0.140 | 4.581 |
| Cu | 0.220 | 3.850 |
| Ti | 2.400 | 3.313 |
| ITO | 2.000 | 0.001 |
| Polypyrrole | 1.700 | 0.300 |

Table 3B

| | Refraction |
|---|---|
| Dielectric | index |
| Methanol | 1.329 |
| (MeOH) | |
| Water | 1.333 |
| Ethanol | 1.361 |
| (EtOH) | |
| Hexane | 1.375 |
| 1-butanol | 1.397 |
| 2-pentanol | 1.404 |
| 1-hexanol | 1.416 |
| 1,3- | 1.438 |
| propanediol | |

For this we use the same deposition housing used in example 2, which is in fact a multitarget enclosure allowing the deposition of 3 distinct materials (FIG. 8) in a single run (the sample remains inside the enclosure all throughout its development phase).

The synthesis of the ITO/Ag/Ti trilayer is done using the process comprising the following steps:
insertion of the transparent solid support in the deposition housing, via the load lock;
pre-sputtering of the ITO target (table 1), the silver target (table 4), then the titanium target (table 4);

| | | |
|---|---|---|
| titanium deposition (table 5) | | Depth of the films |
| silver deposition (table 5) | {close oversize brace} | controlled by the |
| ITO deposition (table 2) | | deposition time |

recovery of the SPR chip, then ready for use.

TABLE 4
Pre-sputtering parameters of the Ti, Ag and
Cu targets

| | | Total | Ratio of | |
|---|---|---|---|---|
| | RF | argon | partial | |
| | Power | pressure | pressures | Target/substrate |
| | P_{RF} | Pₜₒₜ | P_{O2}/P_{Ar} | distance |
| Target | 150 W | 0.008 torr | 0 | 72 mm |
| pre-sputtering: | | | | |
| Ti, Ag, Cu | | | | |

TABLE 5
Deposition parameters of the Ti, Ag and Cu
materials

| | | | Ratio | | |
|---|---|---|---|---|---|
| | | Total | of | | |
| | RF | argon | partial | Target/ | |
| | Power | pressure | pressures | substrate | Deposition |
| | P_{RF} | Pₜₒₜ | P_{O2}P_{Ar} | distance | speed |
| Deposition | 150 W | 0.008 torr | 0 | 72 mm | Ti: 20 nm/min |
| of: | | | | | Ag: 121 nm/ |
| Ti, Ag, | | | | | min |
| Cu | | | | | Cu: 69 nm/min |

The depth of the attachment sub-layer will preferably be around 5 nm, depth from which there is percolation of the first Ti islands, which makes it possible to ensure the effective adhesion of the upper layer of silver.

In this example, the ITO layer is synthesized to:
facilitate covalent grafting of the organic compound(s);
protect the fine layer of gold from mechanical attacks (scratching, . . . ) and/or chemical attacks (oxidation, sulfidation, . . . );
carry out electrochemical measurements;
broaden the electroactive window for use of silver for electrochemical measurements (see Example 6; C. Electrochemical measurements; point C.);
be used for the optical adaptation of the transmittance as a function of the wavelength (43), for example: the role of anti-reflection (FIG. 7) or interference filter.

### Example 4

### Synthesis of the ITO/Cu/Ti Trilayer

A “copper” SPR chip will therefore have a theoretical sensitivity greater than that of a “gold” SPR chip, as a result of the higher numerical value of the imaginary part n″ of the refraction index n of the copper (table 3A). However, in this case as well, since copper oxidizes spontaneously whether in open air or by immersion in the solution to be analyzed, this type of chip therefore cannot be marketed without a layer of TCO being deposited on its surface (ITO in this example).

For this, we use the same deposition housing used in example 3, replacing the silver target with the copper target (FIG. 6). The synthesis of the ITO/Cu/Ti trilayer is done according to the same method as in example 2 (cf. tables 1, 2, 4 and 5).

The advantages of the ITO deposition on the surface of the copper are equivalent to those developed in example 3.

### Example 5

### Synthesis Alternatives to these Preferred Embodiments

A) Substitution of the material used for the attachment layer

a) For the attachment sub-layer of Ti, another material can be substituted such as: Cr, Ni—Cr, Al, Ta or Th.

b) For the attachment sub-layer of Ti, the oxide of the metal used for the plamson response can be substituted, i.e.: AuOx, AgOx, AlOx and/or CuOx.

Indeed, by creating an oxygen concentration gradient in the metal layer with:
maximal x (>0) in the zone in contact with the surface of the substrate. Which makes it possible to create covalent bonds with the oxide making up the substrate (for example, silica SiO₂),
x=0 for the plasmon surface active zone.

### B) Transparent Conductive Oxide Layer Covering the Gold, Silver, Copper or Aluminum

This layer can be made from any material belonging to the TCO (Transparent Conductive Oxide) family (45-50).

### C) Substitution of Glass for the Solid Support

This technique for developing SPR chips at ambient temperature also makes it possible to make these chips on fragile or temperature-sensitive organic substrates, such as Zeonex® transparent polymers (copolyolefin manufactured by NIPPON ZEON), polymethylpentene (TPX), Transphan® (cyclic olefin polymer having a high vitreous transition temperature available from Lofo High Tech Film, GMBH, Germany) or Arton G® or Artone (manufactured by the company Japan Synthetic Rubber Co., Tokyo, Japan) (51).

### Example 6

### Instrumentation

### A) Coupled SPR and Electrochemical Instrumentation

The electrochemical measurements were done using the Autolab 30 potentiostat (Eco Chemie, Utrecht, The Netherlands). The electrode cell used is not the conventional 3 electrode one, but that with two channels of the Autolab SPRINGLE apparatus (Eco Chemie, Utrecht, The Netherlands) allowing simultaneous electrochemical measurement and SPR detection. The configuration of this equipment is described in references 52 and 53. In summary, a polarized laser beam (A=670 nm) illuminates the rear surface of the sensor via a hemispherical lens placed on a prism (BK7 type with a refraction index n=1.52). The reflected light is detected by a photodiode. The incidence angle measured is modified by the use of a mirror oscillating at a frequency of 44 Hz. The SPR curves are recorded with a movement of the mirror from front to back. The reflectance minimum is measured, then averaged.

The measuring apparatus is equipped with an open tank, with a capacity between 20 and 150 μl where the Ag/AgCl reference electrode, the platinum auxiliary electrode and the SPR support with electrical contact on the surface of the sample are submerged. The active surface of the electrode is 0.07 cm².

### B) Results

### a) Influence of the Nature of the Metal (Gold, Silver or Copper) on the Shape of the SPR Signal

The quality of the SPR signal depends critically on several parameters, but essentially on the refraction index of the metal layer and its depth. The refraction index n (characterized by the values of its real part n′ and its imaginary part n″ such that n=n′+in″) of the metal layer has an influence almost as important as its depth (4) on: (i) the angular value of the minimum of the resonance peak (Θ_{SPR}); (ii) the full width at half maximum of this resonance peak (FWHM); (iii) the slopes SL and ST of the resonance peak around Θ_{SPR}, signal head slope and signal tail slope, respectively; (iiii) the reflectance minimum at the resonance angle Θ_{SPR} (Rmin).

During the SPR detection, the movement of the angle Θ_{SPR} (ΔΘ_{SPR}) on the angular scale is measured. For a light beam of given wavelength and a fixed prism, there is an optimal depth of the film for which the intensity of the resonance peak Rₘᵢₙ will be minimal and the full width at half maximum FWHM the narrowest, which will allow a very fine detection of the angular position Θ_{SPR}. These values depend on the refraction indexes (i) of the prism; (ii) of the metal film; (iii) of any layer(s) placed on the surface of the metal film and of course on the respective depth of each of these layers. However, the low adhesion of the film of precious or semiprecious metal to the surface of an oxide (for example, glass) requires the use of an attachment layer (for example, a film of titanium) positioned between the surface of the glass and the metal layer. This attachment layer must be as fine as possible in order to cause minimal disruption of the plasmon signal while also ensuring optical adhesion of the metal layer.

At our working wavelength (λ=670 nm) and with a BK7 prism of index n=1.52, the optimal depths dₘᵢₙ of the metal films used for SPR detection are Au (40 nm); Ag (38 nm); Cu (44 nm); Ti (5 nm). Their respective complex refraction indexes are provided in table 3A. If a metal layer depth d greater than dₘᵢₙ is used, then there will be attenuation of the intensity of the evanescent field due to the reflection of the incident light beam. Rₘᵢₙ will tend toward 1 as d increases. In parallel, the full width at half maximum FWHM and the slopes S_{L} and S_{T} will also be altered.

FIG. 9 shows the experimental and theoretical SPR signals obtained during the immersion in water (n=1.33) of the chips made from multilayers Au/Ti; Ag/Ti; ITO/Au/Ti and ITO/Ag/Ti. As expected, the narrowest SPR peak is obtained with silver (FWHM=2.31⁰; Table 6). The main limitation on the use of the Ag/Ti chip is its chemical instability over time, and particularly when it is immersed in aqueous solutions. A measurement was done with the H₂O/Ag/Ti interface. As we can see, the Θ_{SPR} signal evolves spontaneously toward higher values during the 2 hours of immersion of the chip (FIG. 10). This is the result of the surface formation of silver oxide AgOₓ and/or silver hydroxide Ag(OH).

TABLE 6
Values of theoretical and experimental resonance
angles Θ_{SPR} and full width at half maximum of the SPR peak
(FWHM) for different supports (λ = 670 nm, nₚᵣᵢₛₘ = 1.52,
dielectric = water).

| | exp. and | |
|---|---|---|
| interface | theo. Θ_{SPR}/° | exp FWHM/° |
| Ag/Ti | 66.30 | 2.31 |
| ITO/Ag/Ti | 67.89 | 2.90 |
| Au/Ti | 69.91 | 3.58 |
| ITO/Au/Ti | 72.17 | 6.34 |

It is consequently necessary to protect the silver film from any degradation, in this case chemical here. The deposition of a fine layer of ITO is one solution (see example 3). FIG. 9 presents the SPR signal obtained with the ITO/Ag/Ti heterostructure. Despite the presence of the protective film of ITO 4 nm deep, the narrowness of the SPR peak is preserved (table 6). The position of the signal Θ_{SPR} is offset by 1.5°. However, the stability in water of the chip thus manufactured is excellent (FIG. 10): no significant movement of the value of Θ_{SPR} was detected during 2 hours of immersion.

This ITO deposition can also be done on an Au chip, which does not require particular protection. In this case, a widening of the SPR signal is observed (ΔFWHM=2.8°) as well as an offset (Θ_{SPR}=2.3°) (table 6) in comparison with the Au/Ti bilayer. The chemical stability of the new ITO/Au/Ti heterostructure is comparable to that with silver.

### b) Sensitivity of the SPR Chips

A number of surface plasmon measurement apparatuses are based on the detection and determination of the value of the angular position Θ_{SPR}. In this case, the precision of the measurement depends directly on the narrowness of the SPR peak. Thus, the ITO/Ag/Ti heterostructure combining (i) extreme fineness of the SPR peak, (ii) steep S_{L} and S_{T} slopes, (iii) great chemical stability, will be the ideal chip.

However, other apparatuses integrate, regarding their sensitivity, other parameters such as the amplitude of the movement of the angle Θ_{SPR} as a function of the variation of the refraction index of the reactive medium. It is therefore a combination between the fineness of the SPR peak and its movement amplitude that will define the sensitivity of the signal. FIGS. 11A, 11B and 11C present the evolution of the SPR signals of 3 chips tested: ITO/Ag/Ti; Au/Ti and ITO/Au/Ti when they are in contact with different solutions at increasing refraction indexes (table 3B). The evolution of the position of the angle Θ_{SPR}, the width of the SPR peak and its minimum Rₘᵢₙ for each of the chips are reported in table 7. FIG. 11D shows the theoretical evolution of the SPR signal of an ITO (4 nm)/Cu(44 nm)/Ti(5 nm) chip; results given by the “Windspall” software. In the case of Au/Ti and ITO/Au/Ti devices, one can note a significant Θ_{SPR} variation amplitude, but to the detriment of a widening of the full width at half maximum of the peak and a degradation of its reflectance minimum Rₘᵢₙ. The measurement range of the refraction index of the tested solutions is therefore reduced (n=1.42 at best). Whereas with an ITO/Ag/Ti chip, the more reduced Θ_{SPR} movement amplitude, as well as the low evolution of the width (FWHM) of the SPR signal and of Rₘᵢₙ, makes it possible to precisely measure the refraction indexes of solutions greater than 1.42 (FIG. 12). Moreover, the penetration depth of the evanescent field in the tested solution is doubled compared to that of a gold biochip. The analysis zone of the reactive medium in the vat is thus broader, allowing the characterization of macromolecule(s) grafted to the surface of said support.

TABLE 7
Evolution of the resonance angle Θ_{SPR}, the full
width at half maximum of the SPR peak (FWHM) and the
reflectance minimum Rₘᵢₙ at Θ_{SPR} of different supports as a
function of different dielectric mediums.

| | Θ_{SPR}/° | | | FWHM/° | | | Rmin | | |
|---|---|---|---|---|---|---|---|---|---|
| Dielectric | ITO/Ag/Ti | Au/Ti | ITO/Au/Ti | ITO/Ag/Ti | Au/Ti | ITO/Au/Ti | ITO/Ag/Ti | Au/Ti | ITO/Au/Ti |
| Water | 67.82 | 69.91 | 72.17 | 2.9 | 3.58 | 6.34 | 0.028 | 0.032 | 0.024 |
| Ethanol | 71.62 | 74.31 | 76.98 | 3.04 | 3.87 | 6.44 | 0.031 | 0.046 | 0.014 |
| Hexane | 73.52 | 76.92 | 79.57 | 3.3 | 4.94 | 6.66 | 0.028 | 0.063 | 0 |
| 1-butanol | 77.07 | 81.61 | 85.12 | 3.51 | 5.43 | 6.66 | 0.017 | 0.158 | 0.098 |
| 2-pentanol | 78.39 | 83.50 | 85.91 | 3.57 | 5.72 | 6.82 | 0.017 | 0.253 | 0.345 |
| 1-hexanol | 81.01 | 85.15 | — | 3.57 | 6.01 | — | 0 | 0.592 | — |
| 1,3- | 87.09 | — | — | 3.80 | — | — | 0.598 | — | — |
| propanediol | | | | | | | | | |

Another aspect of the sensitivity of these biochips is their capacity to detect refraction index variations as low as 10⁻⁵ (for example, for biological applications on the nanometric scale). FIG. 13A shows the variation amplitude of the resonance angle (ΔΘ_{SPR}) as a function of the concentration of the NiSO₄ solution tested (molar concentrations between 250.10⁻⁶ mol·L⁻¹ and 50.10⁻³ mol·L⁻¹), the support used being the ITO/Ag/Ti heterostructure. The solution of 50.10⁻³ mol·L⁻¹ leads to a movement of the Θ_{SPR} angle of 0.32°, to which corresponds a variation amplitude Δn of the refraction index of 2.5.10⁻³. The detection limit is obtained with the solution of concentration 400.10⁻⁶ mol. L⁻¹, for which Δn=5,6.10⁻⁵. The detection limits obtained with the Au/Ti and ITO/Au/Ti heterostructures are identical (FIG. 13B).

### c) Electrochemical Measurements

The electrochemical measurements were done using the Autolab 30 potentiostat (Ag/AgCl reference electrode) at the scanning speed of 50 mV/s and with a solution of KCl with a concentration of 0.1 mol·L⁻¹.

The electroactive window of the Au—Ti support is between −1.5V<E<+1.2V (FIG. 14). Beyond a potential of +1.2V, metal Au (degree of oxidation 0) is oxidized in Au⁺ (degree of oxidation+1), which then passes into solution, involving the destruction of the support. In the case of grafting of thiol groups to the surface of this support, the electroactive window is reduced: −0.5V<E<+1.2V. Indeed, the bonds with the thiol groups are destroyed when they are subjected to a cathode potential less than −0.5V (FIG. 14) (54).

The deposition of 4 nm of ITO on an Au/Ti chip makes it possible to widen the electroactive window of the new biochip manufactured: −1.5V<E<+1.9V:

by increasing the polarization at the anodic potentials: the ITO layer prevents the passage into solution of Au⁺, as shown by the voltammogram of FIG. 14;

by increasing the polarization at the cathodic potentials: the ITO layer allowing the grafting of silane functions, measurements at cathodic potentials less than 0.5V are therefore possible (55).

Biochip: ITO/Ag/Ti

The voltammogram obtained with the ITO(4 nm)/Ag(38 nm)/Ti(5 nm) support, as working electrode, is deformed compared to that obtained with the Au(50 nm)/Ti(4 nm) support (FIG. 15). This alteration is caused by the significant electrical resistance of the ITO/Ag/Ti interface. This drawback is easily resolved by increasing the depth of the deposited ITO layer. Indeed, the quality of the voltammogram obtained with the ITO(10 nm)/Ag(38 nm)/Ti(5 nm) support (FIG. 15) is not only identical to that of the classic Au(50 nm)/Ti(4 nm) support, but also makes it possible to widen the working electroactive window: −1.5V<E<+1.9V.

### d) Extension to Preferred Biochips: Polypyrrole/ITO/Ag/Ti Heterostructure

The deposition of a TCO film on the surface of the Au/Ti, Ag/Ti or Cu/Ti supports allows the electrochemical deposition of a fine layer of conductive polymer at their surface. Thus, a film of 5 nm of polypyrrole was deposited on the 3 heterostructures: ITO/Ag/Ti, Au/Ti and ITO/Au/Ti, using the process described in reference 56. FIG. 16 shows the SPR peaks, simulated and measured, for each of the supports submerged in a solution of LiClO₄ with concentration 0.1 mol·L⁻¹. One can see an increase in the full width at half maximum of the SPR peaks of 7.06° and 8.42° for the polypyrrole/Au/Ti and polypyrrole/TIO/Au/Ti supports, respectively, whereas it is only 4.5° for the polypyrrole/TIO/Ag/Ti support. Consequently, this heterostructure is the most suited for measurements to detect and/or monitor reaction(s), since it provides a clearly less distorted SPR signal.

### REFERENCES

(1) Pockrand, I.; Swalen, J. D.; Gordon, J. G.; Philpott, M. R. Surf. Sci. 1978, 74, 237.
(2) Gordon, J. G.; Ernst, S. Surf. Sci 1980, 101, 499.
(3) Swalen, J. D.; Gordon, J. G.; Philpott, M. R.; Brillante, A.; Pockrand, I.; Santo, R. Am. J. Phys. 1980, 48, 669.
(4) Lecaruyer, P.; Canva, M.; Rolland, J. App. Opt. 2007, 46, 2361.
(5) Hutter, E.; Cha, S.; Liu, J.-F.; Park, J.; Yi, J.; Fendler, J. H.; Roy, D. J. Phys. Chem. B 2001, 105, 8-12.
(6) Hutter, E.; Fendler, J. H.; Roy, D. J. Phys. Chem. B 2001, 105, 11159-11168.
(7) Chah, s.; Hutter, E.; Roy, D.; Fendler, J. H.; Yi, J. Chem. Phys. 2001, 272, 127.
(8) Kovacs, G., Optical excitation of surface plasmon-polaritrons in layered media. Wiley ed.; 1982; ‘Vol.’ P. 143-200, p.
(9) Giebel, K.-F.; Bechinger, C.; Herminghaus, S.; Riedel, M.; Leiderer, P.; Weiland, U.; Bastmeyer, M. Biophysical Journal 1999, 76, 509.
(10) Yuan, X.-C.; Ong, B. H.; Tan, Y. G.; Zhang, D. W.; Irawan, R.; Tjin, S. C. J. Opt. A: Pure Appl. Opt. 2006, 8, 959.
(11) Nelson, S. G.; Johnston, K. S.; Yee, S. S. Sens. Actuat. B. 1996, 35/36, 187.
(12) Hanken, D. G.; Jordan, C. E.; Frey, B. L.; Corn, R. M., Marcel Dekker ed.; New York, 1998; ‘Vol.’ 20, p 141-226.
(13) Kooyman, R. P. H.; Kolkman, H.; Van Gent, J.; Greve, J. Anal. Chil. Acta. 1998, 212, 35.
(14) Uznanski, P.; Pecherz, J. J. Appl. Polym. Sci. 2002, 86, 1459.
(15) Winter, G.; Barnes, W. L. App. Phys. Lett. 2006, 88, 051109.
(16) Damos, F. S.; Luz, R. C. S.; Kubota, L. T. Langmuir 2005, 21, (2), 602-609.
(17) Peterlinz, K. A.; georgiadis, R. M.; Herne, T. M.; Tarlov, M. J. J. Am. Chem. Soc. 1997, 119, 3401-3402.
(18) Pyo, H.-B.; Shin, Y.-B.; Kim, M.-G.; Yoon, H. C. Langmuir 2005, 21, 166-171.
(19) Smith, E. A.; Thomas, W. D.; Kiessling, L. L.; Corn, R. M. J. Am. Chem. Soc. 2003, 125, (20) 6140-6148.
(20) Smith, E. A. W., M. J.; Cheng, Y.; Barreira, S. V. P.; Frutos, A. G.; Corn, R. M. Langmuir 2001, 17, (8), 2502-2507.
(21) Jung, L. S.; Campbell, C. T. J. Phys. Chem. B. 2000, 104, 11168-11178.
(22) Fortin, E.; Defontaine, Y.; Mailley, P.; Livache, T.; Szunerits, S. Electronalysis 2005 (23) Guedon, P.; Livache, T.; Martin, F.; Lesbre, F.; Roget, A.; Bidan, G.; Levy, Y. Anal. Chem. 2000, 72, 6003.
(24) Szunerits, S.; Knorr, N.; Calemczuk, R.; Livache, T. Langmuir 2004, 20, (21), 9236-9241.
(25) Van Der Merwe, P. A.; Barclay, A. N. Curr. Opin. Immunol.
(26) Szunerits, S.; Boukherroub, R. Electrochem. Comm. 2006, 8, 439-444.
(27) Szunerits, S.; Coffinier, Y.; Janel, S.; Boukherroub, R. Langmuir 2006, 22, 10716.
(28) Moore et al., Anal Chem. 2007; 79(5):2050-2057).
(29) Arya et al., Anal Biochem. 2007, 15; 363(2):210-218).
(30) T. Le Guillerm, S. Vigneron, X. Castel, G. Legeay, J. Pinel, Elaboration in-situ de couches minces d'ITO par pulvérisation cathodique à température ambiante, 7èmes Journées de Caractérisation Micro-ondes et Matériaux, Toulouse, 03/2002.
(31) S. Vigneron, Elaboration de couches minces d'oxyde d'indium dopé à l'étain par pulvérisation cathodique à température ambiante, Thèse de l'Université de Rennes 1, 8 juillet 2005.
(32) V. Kumar, A. Mansingh, Effect of target-substrate distance on the growth and properties of rf-sputtered indium tin oxide films, J. Appl. Phys. 65(3) (1989) pp. 1270-1280.
(33) V. Singh, Indium tin oxide (ITO) films on flexible substrates for organic light emitting diodes, Proc. of ASID' 06 8-12 Oct. New Delhi.
(34) Y. Shigesato, D. C. Paine, Study of the effect of Sn doping on the electronic transport properties of thin film indium oxide, Appl. Phys. Lett., 62 no 11 (1993) pp. 1268-1270.
(35) H. Morikawa, M. Fujita, Crystallization and electrical property change on the annealing of amorphous indium-oxide and indium-tin-oxide thin films, Thin Solid Films 359 (2000) pp. 61-67.
(36) D. Vaufrey et al. Electrical and optical characteristics of indium tin oxide thin films deposited by cathodic sputtering for top emitting organic electroluminescent devices, Material Science and Engineering C 21 (2002) pp. 265-271.
(37) D. C. Paine et al., A study of low temperature crystallisation of amorphous thin film indium-tin-oxide, J. Appl. Phys. 85 (1999) pp. 8445-8450.
(38) M. Ando et al., Effect of microstrucure on nanocrystallite nucleation and growth in hydrogenated amorphous indium-tin-oxide films, J. Appl. Phys. 93, (2003), pp. 10321038.
(39) L. Li et al., Determination of InP (001) surface reconstruction by STM and infrared spectroscopy of adsorbed hydrogen, Physical Review B, 61 n^{o} 15, (2000), pp. 10223-10228.
(40) K. H. Choi et al., ITO/Ag/ITO multilayer films for the application of a very low resistance transparent electrode, Thin Solid Films, 341 (1999) pp. 152-159.
(41) Y. S. Yung et al., Effects of thermal treatment on the electrical and optical properties of silver-based indium tin oxide/metal/indium tin oxide structures, Thin Solid Films 440 (2003) pp. 278-284.
(42) A. Klöppel et al., Dependence of the electrical and optical behaviour of ITO-silver-ITO multilayers on the silver properties, Thin Solid Films 365 (2000) pp. 139146.
(43) M. Bender et al., Dependence of film composition and thickness on optical and electrical properties of ITO-metal-ITO multilayers, Thin Solid Films, 326, (1998) pp. 67-71.
(44) H. A. Macleod, Thin-Film Optical Filters, Adam Hilger Ltd London 1969.
(45) B. G. Lewis and D. C. Paine, Applications and Processing of Transparent Conducting Oxides, MRS Bulletin, 10 (2000) pp. 22-27.
(46) H. Kawazoe, H. Yanagi, K. Ueda, and H. Hosono, Transparent p-Type Conducting Oxides: Design and Fabrication of p-n Heterojunctions, MRS Bulletin, 10 (2000) pp. 28-36.
(47) T. Minami, New n-Type Transparent Conducting Oxides, MRS Bulletin, 10 (2000) pp. 38-44.
(48) A. J. Freeman, K. R. Poeppelmeier, T. O. Mason, R. P. H. Chang, and T. J. Marks, Chemical and Thin Film Strategies for New Transparent Conducting Oxides, MRS Bulletin, 10 (2000) pp. 45-51.
(49) R. G. Gordon, Criteria for Choosing Transparent Conductors, MRS Bulletin, 10 (2000) pp. 52-57.
(50) T. J. Coutts, D. L. Young, and X. Li, Characterization of Transparent Conducting Oxides, MRS Bulletin, 10 (2000) pp. 58-65.
(51) J. H. Schut, New Cyclic Olephins, Plastics Technology Online, Gardner 2005.
(52) Wink, T.; Van Zuilen, S. J.; Bult, A.; van Bennekom, W. P. Chem. Comm. 1998, 70, 827-832.
(53) Kooyman, R. P. H.; Lenferink, A. T. M.; Eenink, R. G.; Greve, J. Anal. Chem. 1991, 63, 83-85.
(54) G. Wittstock, R. Hesse, W. Schuhmann, Electroanalysis, 2005, 9, 746
(55) S. Szunerits, Rabah Boukherroub, Electrochem. Comun. 2006, 8, 439-444
(56) Szunerits, S.; Knorr, N.; Calemczuk, R.; Livache, T. Langmuir 2004, 20(21), 9236-9241

1. A solid support, characterized in that it comprises a transparent solid support coated partially or completely with:
at least one layer of at least one metal to form a solid support that can be used for detection by SPR;
at least one transparent conductive oxide (TCO) layer;
and, if necessary, an attachment layer.

2. The solid support according to claim 1, characterized in that said TCO layer is formed by at least one transparent conductive oxide (TCO) layer to form a solid support that can be used for detection by SPR and/or for electrochemical detection.

3. The solid support according to claim 1 or 2, characterized in that said TCO layer comprises at least one transparent conductive oxide chosen from the group made up of In₂O₃₋ₓ with 0<x<3; ZnO₁₋ₓ with 0<x<1; SnO₂₋ₓ with 0<x<2; CdO₁₋ₓ with 0<x<1; Ga₂O₃₋ₓ with 0<x<3; Tl₂O₃₋ₓ with 0<x<3; PbO₂₋ₓ with 0<x<2; Sb₂O₅₋ₓ with 0<x<5; MgO₁₋ₓ with 0<x<1 and TiO₂₋ₓ with 0<x<2.

4. The solid support according to claim 3, characterized in that said TCO layer comprises at least one transparent conductive oxide chosen from the group made up of In₂O₃; ZnO, SnO₂; CdO; Ga₂O₃; Tl₂O₃; PbO₂; Sb₂O₅; MgO and TiO₂.

5. The solid support according to one of claims 1 to 4, characterized in that said TCO layer comprises at least one transparent conductive oxide made up of a combination of at least two binary oxides.

6. The solid support according to one of claims 1 to 5, characterized in that said TCO layer also comprises a component capable of doping the TCO.

7. The solid support according to one of claims 1 to 6, characterized in that said TCO layer is a layer comprising indium oxide In₂O₃.

8. The solid support according to one of claims 1 to 7, characterized in that said TCO layer is a layer comprising tin-doped indium oxide (ITO), preferably synthesized from a target material made up of a mixture 90% In₂O₃ and 10% SnO₂ by mass.

9. The solid support according to one of claims 1 to 8, characterized in that said TCO layer is a layer comprising tin-doped indium oxide (ITO) deposited at ambient temperature and with mainly amorphous structure.

10. The solid support according to one of claims 1 to 9, characterized in that said TCO layer has a depth between 3 nm and 200 nm.

11. The solid support according to claim 10, characterized in that said TCO layer has a depth between 4 nm and 10 nm.

12. The solid support according to one of claims 1 to 11, characterized in that said layer made up of at least one metal is a layer whereof the metal is chosen from the group made up of gold, silver, copper and aluminum or by any combination of these metals or of their respective alloys.

13. The solid support according to one of claims 1 to 12, characterized in that said layer made up of at least one metal has a depth between 10 nm and 200 nm, preferably between 30 and 50 nm.

14. The solid support according to one of claims 1 to 13, characterized in that said solid support is coated with an attachment layer before said layer made up of at least one metal, preferably with a depth between 1 nm and 10 nm, 5 nm±1 nm being the preferred depth.

15. The solid support according to claim 14, characterized in that said attachment layer is a metal layer whereof the metal is chosen from the group made up of titanium, chrome, nickel, tantalum, molybdenum, thorium, copper, aluminum or tin or by any combination of these metals or of their respective alloys, oxides and/or hydroxides.

16. The solid support according to claim 14, characterized in that said attachment layer is a metal oxide MOx layer, with oxygen gradient, with M designating at least one metal chosen from the group of gold, silver, copper and aluminum, or by any combination of these metals or of their respective alloys.

17. The solid support according to one of claims 14 to 16, characterized in that said attachment layer is preferably a layer of titanium.

18. The solid support according to one of claims 1 to 17, characterized in that said solid support is made up of at least one organic or inorganic transparent material or of a combination of transparent materials such as glass or transparent solid polymers such as polymethylpentene (TPX), polyethylene, polyethylene terephthalate (PET), polycarbonate, preferably glass.

19. The solid support according to one of claims 1 to 18, characterized in that said layer of at least one metal to form a solid support that can be used for detection by SPR is a layer made up of metal nanoparticles.

20. The solid support according to claim 19, characterized in that it can be chosen among the supports shown in FIGS. 20 and 21.

21. The solid support according to one of claims 1 to 19, characterized in that said transparent conductive oxide (TCO) layer is coated with a layer made up of metal nanoparticles, preferably as shown in FIG. 17.

22. The solid support according to one of claims 1 to 19, characterized in that said transparent conductive oxide (TCO) layer is coated with a layer made up of metal nanoparticles, the latter layer of metal nanoparticles itself being coated with a TCO layer, preferably as shown in FIG. 18.

23. The solid support according to claim 22, as shown in FIG. 19 where n is between 2 and 5 (inclusive).

24. The solid support according to one of claims 1 to 16, characterized in that at least one TCO layer, preferably the last TCO layer deposited if the support contains several, has hydroxyl groups, chemically active if necessary.

25. The solid support according to claim 24, characterized in that said hydroxyl groups and/or the functional groups having reacted with said hydroxyl groups can be desorbed from said TCO layer by exposure to ultraviolet radiation.

26. A process for manufacturing a solid support for detection by surface plasmon resonance (SPR) and/or by electrochemical methods, characterized in that it comprises the following steps:
the deposition on at least a same surface of the solid support and superimposed,
of at least one layer made up of at least one metal to form a solid support that can be used for detection by SPR; and
of at least one transparent conductive oxide (TCO) layer, preferably said TCO layer is made up of at least one transparent conductive oxide (TCO) layer to form a solid support that can be used for detection by SPR and/or for electrochemical detection.

27. The manufacturing process according to claim 26, characterized in that the deposition of the TCO layer is done in a vacuum chamber, preferably provided with a residual pressure between 10⁻⁵ and 10⁻⁷ mbar or less.

28. The manufacturing process according to claim 26 or 27, characterized in that the deposition of the TCO layer is done under partial vacuum in the presence of at least one rare gas, preferably argon, or in the presence of a mixture of a rare gas (preferably argon) and a gas containing the oxygen element (preferably dioxygen), preferably at a pressure of about 0.012 mbar of rare gas/dioxygen mixture, preferably with a p_{O2}/p_{Ar} ratio equal to about 5.1.10⁻⁴.

29. The manufacturing process using cathode sputtering according to one of claims 26 to 28, characterized in that the cathode sputtering housing comprises at least one generator, preferably radiofrequency (RF), the radiofrequency power used for the deposition preferably being between 0.1 W/cm² and 4 W/cm² for a target-substrate distance between 10 mm and 150 mm.

30. The manufacturing process according to one of claims 26 to 29, characterized in that the depth of said TCO layer is controlled by the deposition duration, preferably at a speed of 0.6 nm/min at the power of 0.86 W/cm² for a target-substrate distance of 78 mm.

31. The manufacturing process according to one of claims 26 to 30, characterized in that said TCO layer comprises at least one transparent conductive oxide chosen from the group made up of In₂O₃₋ₓ with 0<x<3; ZnO₁₋ₓ with 0<x<1; SnO₂₋ₓ with 0<x<2; CdO₁₋ₓ with 0<x<1; Ga₂O₃₋ₓ with 0<x<3; Tl₂O₃₋ₓ with 0<x<3; PbO₂₋ₓ with 0<x<2; Sb₂O₅₋ₓ with 0<x<5; MgO₁₋ₓ with 0<x<1 and TiO₂₋ₓ with 0<x<2, preferably chosen among In₂O₃; ZnO; SnO₂; CdO; Ga₂O₃; Tl₂O₃; PbO₂; Sb₂O₅; MgO; TiO₂.

32. The manufacturing process according to one of claims 26 to 31, characterized in that said TCO layer comprises at least one oxide made up of a combination of at least two binary oxides.

33. The manufacturing process according to one of claims 26 to 32, characterized in that said TCO layer comprises any combination of the TCOs with a component capable of doping said TCOs such as Sn for In₂O₃.

34. The manufacturing process according to one of claims 26 to 33, characterized in that said TCO layer is a layer comprising indium oxide In₂O₃.

35. The manufacturing process according to one of claims 26 to 34, characterized in that said TCO layer is a layer comprising tin-doped indium oxide (ITO), preferably synthesized from a target material made up of a mixture 90% In₂O₃ and 10% SnO₂ by mass.

36. The manufacturing process according to one of claims 26 to 35, characterized in that said TCO layer is a layer comprising tin-doped indium oxide (ITO) deposited at ambient temperature and with a mainly amorphous structure.

37. The manufacturing process according to one of claims 26 to 36, characterized in that said TCO layer has a depth between 3 nm and 200 nm, preferably a depth between 4 nm and 10 nm.

38. The manufacturing process according to one of claims 26 to 37, characterized in that said layer made up of at least one metal is a layer whereof the metal is chosen from the group made up of gold, silver, copper and aluminum or any combination of these metals or of their respective alloys.

39. The manufacturing process according to one of claims 26 to 38, characterized in that said layer made up of at least one metal has a depth between 10 nm and 200 nm, preferably between 30 and 50 nm.

40. The manufacturing process according to one of claims 26 to 39, characterized in that said solid support is previously coated with an attachment layer.

41. The manufacturing process according to claim 40, characterized in that said attachment layer is a metal layer whereof the metal is chosen from the group made up of titanium, chrome, nickel, tantalum, molybdenum, thorium, copper, aluminum, tin, or by any combination of these metals or of their respective alloys, oxides and/or hydroxides.

42. The manufacturing process according to claim 40, characterized in that said attachment layer is a layer of metal oxide MOx, with oxygen gradient, with M designating at least one metal chosen in the group made up of gold, silver, copper and aluminum, or by any combination of these metals or of their respective alloys.

43. The manufacturing process according to one of claims 40 to 42, characterized in that said attachment layer is a layer of titanium.

44. The manufacturing process according to one of claims 40 to 43, characterized in that said attachment layer has a depth between 1 and 10 nm, before the deposition of said layer made up of at least one metal, preferably with a depth of 5 nm±1 nm.

45. The manufacturing process according to one of claims 40 to 44, characterized in that said solid support is previously coated with an attachment layer and said layer made up of at least one metal, before the deposition of said TCO layer.

46. The manufacturing process according to one of claims 40 to 45, characterized in that if necessary, said attachment layer and said layer made up of at least one metal is (are) also deposited by cathode sputtering.

47. The manufacturing process according to claim 46, characterized in that, if necessary, said attachment layer, said layer made up of at least one metal and said TCO layer are successively deposited on said solid support by cathode sputtering within a same device comprising an enclosure provided with a system of at least two targets, one of which is made up of the metal or alloy used to develop said layer made up of at least one metal, and the other of which is made up of the material used to develop said TCO layer, and, if applicable, of a target made of metal or alloy used to develop said attachment layer, and, if necessary, any useful target, said device being provided with at least one vacuum pump to create a partial vacuum in the enclosure and at least one controlled inlet for rare gas, preferably argon, and, if necessary, additional controlled inlets for reactive gas(es), preferably a carrier gas of the oxygen element, preferably dioxygen, and/or of at least one controlled inlet for a pre-established mixture of rare gas and reactive gas(es), preferably a carrier gas of the oxygen element, preferably dioxygen.

48. The manufacturing process according to one of claims 26 to 47, characterized in that said solid support is made up of at least one organic or inorganic transparent material or a combination of transparent materials.

49. The manufacturing process according to claim 48, characterized in that said solid support(s) is (are) chosen among glass or transparent solid polymers such as polymethylpentene (TPX), polyethylene, polyethylene terephthalate (PET), polycarbonate, preferably glass.

50. A use of a support according to one of claims 1 to 25, or capable of being obtained using a method according to one of claims 26 to 49, for the determination of at least one organic or mineral compound in a sample or for the monitoring of at least one reaction in a complex mixture by SPR and/or electrochemical detection.

51. The use of a support according to claim 50 for the detection in a sample of chemical or mineral compounds, comprising in particular polymers or heavy metals, organic or biological compounds or structures comprising in particular nucleic acids, polypeptides or proteins, carbon hydrates, organic particles such as liposomes or vesicles, inorganic particles (such as micro- or nanospheres, cellular organelles or cells).

52. A kit for determining the presence and/or quantity of at least one compound or for the monitoring of at least one reaction in a sample by SPR and/or by electrochemistry, characterized in that it comprises a support according to one of claims 1 to 25 or a support capable of being obtained using a process according to one of claims 26 to 49.

53. A diagnostic or analysis device comprising a support according to one of claims 1 to 25, or capable of being obtained using a process according to one of claims 26 to 49.

54. A device comprising an enclosure provided with a system of at least two targets, one of which is made up of the metal or alloy used to develop said layer made up of at least one metal, and the other of which is made up of the material used to develop said TCO layer, and, if applicable, of a target made of metal or alloy used to develop said attachment layer, and, if necessary, any useful target as defined in one of claims 1 to 49, said device being provided with at least one vacuum pump to create a partial vacuum in the enclosure and at least one controlled inlet for rare gas, preferably argon, and, if necessary, additional controlled inlets for reactive gas(es), preferably a carrier gas of the oxygen element, preferably dioxygen and/or of at least one controlled inlet for a pre-established mixture of rare gas and reactive gas(es), preferably a carrier gas of the oxygen element, preferably dioxygen.
